(19) United States
(12) Patent Application Publication
(10) Pub. No.: US 2014/0242113 A1
(43) Pub. Date: Aug. 28, 2014

(54) LIQUID VACCINE PREPARATIONS

(71) Applicant: Leonard P. Ruiz, JR., Shoreview, MN (US)

(72) Inventor: Leonard P. Ruiz, JR., Shoreview, MN (US)

(21) Appl. No.: 14/114,698

(22) PCT Filed: Apr. 27, 2012
(86) PCT No.: PCT/US2012/035572

§ 371 Date: May 12, 2014

Related U.S. Application Data
(60) Provisional application No. 61/480,118, filed on Apr. 28, 2011.

(51) Int. Cl. A61K 47/46; A61K 39/13; A61K 47/44; A61K 47/24; A61K 47/02; A61K 39/15; A61K 39/00

(52) U.S. Cl. CPC A61K 47/46; A61K 39/15; A61K 39/13; A61K 39/00; A61K 47/24; A61K 47/02; A61K 47/44; A61K 2039/5254; A61K 2039/52; A61K 2039/51
USPC 424/215.1; 424/204.1; 424/217.1; 424/261.1; 424/265.1

(57) ABSTRACT

This document provides methods and materials involved in making and using liquid vaccine preparations for oral administration. For example, methods and materials for making and using liquid vaccine preparations for oral administration that include a lyophilized or dried vaccine component (e.g., a lyophilized pathogenic agent such as a lyophilized rotavirus preparation) and a liquid edible oil composition (e.g., a liquid edible oil composition containing one or more medium chain triglycerides) are provided. In some cases, liquid vaccine preparations that include a buffer component (e.g., CaCO₃) are provided.

### CROSS-REFERENCE TO RELATED APPLICATION

This application claims priority to U.S. Provisional Patent Application Ser. No. 61/480,118, filed Apr. 28, 2011, which incorporated herein by reference in its entirety.

### BACKGROUND

1. Technical Field

This document relates to methods and materials involved in making and using liquid vaccine preparations for oral administration. For example, this document relates to methods and materials for making and using liquid vaccine preparations for oral administration that include a lyophilized or dried vaccine component (e.g., a lyophilized pathogenic agent such as a lyophilized rotavirus preparation) and a liquid edible oil composition (e.g., a liquid edible oil composition containing one or more medium chain triglycerides). In some cases, a liquid vaccine preparation can include a buffer component (e.g., CaCO₃).

2. Background Information

In general, vaccines are biological preparations designed to improve a mammal's immunity to a particular pathogen. A vaccine can contain one or more components that resemble a pathogen capable of causing disease and is often made from a weakened form of the pathogen. The vaccine components are designed to stimulate the mammal's immune system to recognize the components of the vaccine and/or the pathogen they resemble as foreign, thereby enabling the mammal's immune system to destroy the pathogen quickly when encountered in the future.

### SUMMARY

This document provides methods and materials related to liquid vaccine preparations. For example, this document provides liquid vaccine preparations as well as methods and materials for making and using liquid vaccine preparations. A liquid vaccine preparation can include a lyophilized or dry vaccine component (e.g., a lyophilized Rotavirus vaccine such as lyophilized RotaShield) and a liquid edible oil composition (e.g., a liquid edible oil composition containing one or more medium chain triglycerides). As described herein, dry vaccine components such as lyophilized rotaviruses can be formulated together with a liquid edible oil composition to create a suspension that allows the vaccine components to be shipped, stored, and delivered to a mammal in liquid form with the vaccine components retaining an effective titer level without the need of being maintained at temperatures less than 4° C. (e.g., refrigerator or freezer temperatures). For example, a liquid vaccine preparation provided herein can be maintained at room temperature for extended periods of time (e.g., greater than 30 days) in a stable manner such that an effective titer level (e.g., titer of at least 1×10⁴ plaque forming units (PFUs) or fluorescent focus units (FFUs) for viruses or colony forming units (CFUs) for microorganisms per mL) is retained. Having the ability to provide vaccine preparations in liquid form that remain stable at room temperatures can allow users to deliver the vaccine preparation as a liquid without the need for the user or others to reconstitute or otherwise formulate a dried composition into a liquid composition at the point of care or at a point along delivery to the point of care. In some cases, having the ability to provide vaccine preparations in liquid form that remain stable at room temperatures can allow one to avoid the need for refrigeration conditions for distribution and storage of the vaccine preparations.

In some cases, a vaccine preparation provided herein can include a buffer component (e.g., CaCO₃). For example, a vaccine component configured for oral delivery can be formulated together with one or more buffers in a liquid edible oil composition. A buffer component of a vaccine preparation provided herein can be included in an amount effective to reduce the effect of gastric fluid on the vaccine component, thereby increasing the viability of the vaccine component following oral administration to a subject. As described herein, dry vaccine components can be formulated together with one or more dry or powdered buffers in a liquid edible oil composition to create a suspension with minimal or no interaction between the vaccine components and the buffer components within the suspension, thereby allowing the vaccine components to remain stable for extended periods of time (e.g., greater than 30 days) at room temperatures or warmer without the need for separately housing the buffer components in a container separate from a container housing vaccine components. Thus, having the ability to provide vaccine preparations in liquid form that remain stable at room temperatures and include one or more buffer components can allow manufacturers to provide users with a ready to use vaccine preparation in a container that does not require a chamber for the vaccine components that is separate from a chamber for the buffer components.

In general, one aspect of this document features a liquid vaccine preparation for oral delivery to a mammal. The preparation comprises, or consists essentially of, an edible oil in liquid form and viable pathogens present within the edible oil in the form of a suspension, wherein the vaccine preparation undergoes no more than a 50 percent reduction in viability of the viable pathogens when stored at 37° C. for 30 days. In various embodiments, the viable pathogens can include a viable virus, a viable microorganism, or a viable parasite. The preparation can comprise a dry or lyophilized viable pathogen. The preparation can comprise a buffer component present within the edible oil in the fowl of a suspension. The vaccine preparation can undergo no more than a 25 percent reduction in viability of the viable pathogens when stored at 37° C. for 30 days. The vaccine preparation can undergo no more than a 10 percent reduction in viability of the viable pathogens when stored at 37° C. for 30 days. The vaccine preparation can undergo no more than a 5 percent reduction in viability of the viable pathogens when stored at 37° C. for 30 days. The vaccine preparation can undergo no more than a 1 percent reduction in viability of the viable pathogens when stored at 37° C. for 30 days.

In another aspect, this document features a vaccine preparation comprising, or consisting essentially of, an edible oil in liquid form, a buffer component present within the edible oil in the form of a suspension, and viable pathogens (e.g., viable viruses, viable microorganisms, or viable parasites) present within the edible oil in the form of a suspension, wherein (a) the vaccine preparation has a titer of at least a minimum pathogen titer per mL of vaccine preparation or (b) the vaccine preparation undergoes no more than a 50 percent reduction in viability of the viable pathogens when stored at 37° C. for 30 days. The edible oil can comprise medium chain triglycerides. Suitable fatty acids of the medium chain triglycerides can include, for example, caproic acid, caprylic acid, capric acid, and/or lauric acid. At least 40 percent of the edible oil can comprise medium chain triglycerides. At least 50 percent of the edible oil can comprise medium chain triglycerides. At least 60 percent of the edible oil can comprise medium chain triglycerides. At least 65 percent of the edible oil can comprise medium chain triglycerides. The edible oil can comprise a coconut oil, a corn oil, a cottonseed oil, an olive oil, a palm oil, a peanut oil, a rapeseed oil, a safflower oil, a sesame oil, a soybean oil, or a sunflower oil. The edible oil can be an olive oil, a soybean oil, or a sunflower oil. The buffer component can comprise calcium carbonate, sodium carbonate, potassium carbonate, sodium bicarbonate, potassium bicarbonate, calcium bicarbonate, aluminum hydroxide, or magnesium hydroxide. The buffer component can be sodium bicarbonate or calcium carbonate. The viable pathogens can include a viable virus such as an attenuated rotavirus. Alternatively, viable viruses can be a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4. The viable viruses can be a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4. The viable viruses can be attenuated polioviruses. The viable viruses can comprise lyophilized viable viruses. In embodiments in which the viable pathogens include a viable virus, the vaccine preparation can have a viral titer of at least 3.5×10⁵ PFUs or FFUs per mL of vaccine preparation. The vaccine preparation can have a viral titer of at least 8×10⁵ PFUs or FFUs per mL of vaccine preparation. The vaccine preparation can lack water (or contain no more than 5%, 4%, 3%, 2%, or 1% water). In some cases, the viable viruses of the vaccine preparation can be lyophilized viable viruses that can be combined with the edible oil. In some cases, the buffer component of the vaccine preparation can be a powder form of the buffer component that can be combined with the edible oil. The vaccine preparation can lack dissolved viable viruses. The vaccine preparation can lack a dissolved buffer component. The vaccine preparation can lack dissolved viable viruses and a dissolved buffer component. The vaccine preparation can comprise a colorant. The vaccine preparation can comprise a colorant, flavoring, or sweetener. The vaccine preparation can be in the form of a liquid containing a suspension and adapted for oral administration.

In another aspect, this document features a method for making a vaccine preparation in liquid form. The method comprises, or consists essentially of, combining dry viable pathogens viruses (e.g., dry viable viruses, dry viable microorganisms, or dry viable parasites) with an edible oil in liquid form under conditions wherein the dry viable pathogens form a suspension within the edible oil, thereby forming the vaccine preparation in liquid form, and wherein (a) the vaccine preparation has a titer of at least a minimum pathogen titer per mL of vaccine preparation or (b) the vaccine preparation undergoes no more than a 50 percent reduction in viability of the viable pathogens when stored at 37° C. for 30 days. The dry viable pathogens can include a dry viable virus such as a dry attenuated rotavirus. Alternatively, dry viable viruses can be a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4. The viable viruses can be a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4. The dry viable viruses can be dry attenuated polioviruses. The dry viable viruses can comprise lyophilized viable viruses. In embodiments in which the dry viable pathogens include dry viable viruses, the vaccine preparation can have a viral titer of at least 3.5×10⁵ PFUs or FFUs per mL of vaccine preparation. The vaccine preparation can have a viral titer of at least 8×10⁵ PFUs or FFUs per mL of vaccine preparation. The edible oil can comprise medium chain triglycerides. The fatty acids of the medium chain triglycerides can include, for example, caproic acid, caprylic acid, capric acid, and/or lauric acid. At least 40 percent of the edible oil can comprise medium chain triglycerides. At least 50 percent of the edible oil can comprise medium chain triglycerides. At least 60 percent of the edible oil can comprise medium chain triglycerides. At least 65 percent of the edible oil can comprise medium chain triglycerides. The edible oil can comprise a coconut oil, a corn oil, a cottonseed oil, an olive oil, a palm oil, a peanut oil, a rapeseed oil, a safflower oil, a sesame oil, a soybean oil, or a sunflower oil. The edible oil can be an olive oil, a soybean oil, or a sunflower oil. The method can comprise combining a dry buffer component with the edible oil under conditions wherein the dry buffer component forms a suspension within the edible oil. The dry buffer component can comprise calcium carbonate, sodium carbonate, potassium carbonate, sodium bicarbonate, potassium bicarbonate, calcium bicarbonate, aluminum hydroxide, magnesium hydroxide, or a mixture thereof. The buffer component can be sodium bicarbonate, calcium carbonate, or a mixture thereof. The buffer component can be in powder form prior to combining the dry buffer component with the edible oil. The vaccine preparation can lack water (or contains no more than 5%, 4%, 3%, 2%, or 1% water). The method can lack a step of adding water to the vaccine preparation. The vaccine preparation can lack dissolved viable viruses. The vaccine preparation can lack a dissolved buffer component. The vaccine preparation can lack dissolved viable viruses and a dissolved buffer component. The method can comprise combining a colorant with the edible oil. The method can comprise combining flavoring or sweetener with the edible oil. The method can comprise inserting the vaccine preparation in the form of a liquid containing a suspension into a container adapted for oral delivery to a mammal. The container can be an oral syringe. The container can be an oral syringe-like container. The mammal can be a human. The mammal can be a human child less than three years of age.

In another aspect, this document features a method for vaccinating a mammal. The method comprises, or consists essentially of, orally administering a vaccine preparation in liquid form to a mammal, wherein the vaccine preparation comprises an edible oil in liquid form and viable pathogens (e.g., viable viruses, viable microorganisms, or viable parasites) present within the edible oil in the form of a suspension, wherein (a) the vaccine preparation has a titer of at least a minimum pathogen titer FFUs per mL of vaccine preparation or (b) the vaccine preparation undergoes no more than a 50 percent reduction in viability of the viable pathogens when stored at 37° C. for 30 days. The edible oil can comprise medium chain triglycerides. The fatty acids of the medium chain triglycerides can include, for example, caproic acid, caprylic acid, capric acid, and/or lauric acid. At least 40 percent of the edible oil can comprise medium chain triglycerides. At least 50 percent of the edible oil can comprise medium chain triglycerides. At least 60 percent of the edible oil can comprise medium chain triglycerides. At least 65 percent of the edible oil can comprise medium chain triglycerides. The edible oil can comprise a coconut oil, a corn oil, a cottonseed oil, an olive oil, a palm oil, a peanut oil, a rapeseed oil, a safflower oil, a sesame oil, a soybean oil, a sunflower oil, or a mixture thereof. The edible oil can be an olive oil, a soybean oil, a sunflower oil, or a mixture thereof. The vaccine preparation can comprise a buffer component present within the edible oil in the form of a suspension. The buffer component can comprise calcium carbonate, sodium carbonate, potassium carbonate, sodium bicarbonate, potassium bicarbonate, calcium bicarbonate, aluminum hydroxide, magnesium hydroxide, or a mixture thereof. The buffer component can be sodium bicarbonate, calcium carbonate, or a mixture thereof. In some cases, the buffer component of the vaccine preparation was a powder form of the buffer component that was combined with the edible oil. The viable pathogens can include a viable virus such as an attenuated rotavirus. Alternatively, viable viruses can be a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4. The viable viruses can be a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4. The viable viruses can be attenuated polioviruses. The viable viruses can comprise lyophilized viable viruses. In embodiments in which the viable pathogens include viable viruses, the vaccine preparation can have a viral titer of at least 3.5×10⁵ PFUs or FFUs per mL of vaccine preparation. The vaccine preparation can have a viral titer of at least 8×10⁵ PFUs or FFUs per mL of vaccine preparation. The vaccine preparation can lack water (or contains no more than 5%, 4%, 3%, 2%, or 1% water). In some cases, the viable viruses of the vaccine preparation can be lyophilized viable viruses that can be combined with the edible oil. The vaccine preparation can lack dissolved viable viruses. The vaccine preparation can lack a dissolved buffer component. The vaccine preparation can lack dissolved viable viruses and a dissolved buffer component. The vaccine preparation can comprise a colorant. The vaccine preparation can comprise flavoring or sweetener. The mammal can be a human. The mammal can be a human child less than three years of age. The vaccine preparation can be stored at room temperature for a period of time before being administered to the mammal, wherein the period of time is at least 15 days. The period of time can be at least 30 days. The vaccine preparation can be stored at a temperature between 15° C. and 30° C. for a period of time before being administered to the mammal, wherein the period of time is at least five days. The period of time can be at least ten days. The period of time can be at least 30 days. The temperature can be between 18° C. and 25° C. The period of time can be at least ten days. The period of time can be at least 30 days.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

This document provides methods and materials related to liquid vaccine preparations. For example, this document provides liquid vaccine preparations as well as methods and materials for making and using liquid vaccine preparations. A liquid vaccine preparation can be formulated for oral delivery and can include a lyophilized or dry vaccine component (e.g., a lyophilized Rotavirus vaccine such as lyophilized RotaShield) and a liquid edible oil composition (e.g., a liquid edible oil composition containing one or more medium chain triglycerides).

A liquid vaccine preparation provided herein can include any appropriate vaccine component. In general, a vaccine component is designed to contain one or more antigens of a pathogen for which a mammal (e.g., a human) is to be immunized. A pathogen can be a virus, a microorganism, or a parasite. In some cases, a vaccine component can be a viable virus, a viable microorganism, or a viable parasite for which a mammal (e.g., a human) is to be immunized. For example, a liquid vaccine preparation provided herein can include a viable virus preparation as a vaccine component. Examples of viable virus preparations include, without limitation, viable rotavirus (e.g., rhesus, rhesus/human reassortant, ovine, bovine, bovine/human reassortant, or human rotaviruses), polio virus, enterovirus (e.g., enterovirus 71), rabies virus, ebola virus, adenovirus, poxvirus, influenza virus, herpes viruses, or norovirus preparations. In some cases, a liquid vaccine preparation provided herein can include a viable microorganism preparation as a vaccine component. Examples of viable microorganism preparations include, without limitation, viable Salmonella strains (e.g., Salmonella typhi), Helicobacter pylori, Clostridium difficile (e.g., toxin A+ B+ strains of Clostridium difficile), Rhodococcus equi, Vibrio cholera, Escherichia coli, Shigella strains, Mycobacterium tuberculosis (e.g., BCG vaccine components), or Listeria, Lactobacilli microorganism preparations. In some cases, a liquid vaccine preparation provided herein can include a viable parasite preparation as a vaccine component. Examples of viable parasite preparations include, without limitation, viable Cryptosporidium parvum or Onchocerca volvulus (river blindness) parasite preparations.

In some cases, a vaccine component can be a viable virus, a viable microorganism, or a viable parasite that is attenuated. For example, a viable, attenuated viral preparation can be used as a vaccine component of liquid vaccine preparation provided herein. In some cases, a vaccine component can be a viable virus, a viable microorganism, or a viable parasite that is genetically altered to express one or more antigens (e.g., polypeptides such as an amyloid β-peptide for oral immunization to aid in treating Alzheimer's Disease) of a pathogen for which a mammal (e.g., a human) is to be immunized. For example, an adenovirus, poxvirus, influenza virus, herpes virus, or poliovirus can be genetically altered to express one or more antigens of another virus or pathogenic bacterial species (e.g., E. coli). In such cases, a viable preparation of the genetically modified virus can be used as a vaccine component of liquid vaccine preparation provided herein. In some cases, genetically modified Salmonella, Escherichia coli, Listeria, Shigella, or Lactobacilli microorganism can be uses as vectors to orally deliver antigens into the gut-associated lymphoid tissue (GALT). Any appropriate molecular biology technique can be used to engineer a virus, microorganism, or parasite to express one or more polypeptides (e.g., polypeptides from a pathogen). For example, common molecular biology techniques such as molecular cloning techniques can be used to engineer a virus, microorganism, or parasite to express one or more polypeptides from a pathogen.

In some cases, a viable virus preparation, viable microorganism preparation, or viable parasite preparation of a liquid vaccine preparation provided herein can be lyophilized or used in an otherwise dry form. For example, a viable virus preparation that is in lyophilized form can be used as a vaccine component of liquid vaccine preparation provided herein.

Any appropriate lyophilization or drying technique can be used to make a dry form of a viable virus preparation, a viable microorganism preparation, or a viable parasite preparation. For example, standard freeze drying techniques can be used to make a lyophilized preparation of viable viruses, viable microorganisms, or viable parasites. In some cases, drying techniques such as those described in U.S. Pat. Nos. 4,622,222; 5,024,836; 5,716,615; and 5,895,648 can be used to make a dry (e.g., lyophilized) form of a viable virus preparation, a viable microorganism preparation, or a viable parasite preparation.

In some cases, a lyophilized preparation of viable viruses, viable microorganisms, or viable parasites used as a vaccine component of a liquid vaccine preparation provided herein can contain no more than 10 percent of water (e.g., no more than 9 percent, no more than 8 percent, no more than 7 percent, no more than 6 percent, no more than 5 percent, no more than 4 percent, no more than 3 percent, no more than 2 percent, or no more than 1 percent of water).

A liquid vaccine preparation provided herein can include a liquid edible oil composition. In some cases, a liquid vaccine preparation provided herein can include a liquid edible oil composition and a lyophilized vaccine component of viable viruses, microorganisms, or parasites that is present, at least in part, in the edible oil composition as a suspension. In some cases, no more than 5 percent (e.g., no more than 4 percent, no more than 3 percent, no more than 2 percent, or no more than 1 percent) of a lyophilized vaccine component of viable viruses, microorganisms, or parasites is dissolved in the edible oil composition of the liquid vaccine preparation. In some cases, at least 90 percent (e.g., at least 91 percent, at least 92 percent, at least 93 percent, at least 94 percent, at least 95 percent, at least 96 percent, at least 97 percent, at least 98 percent, or at least 99 percent) of a lyophilized vaccine component of viable viruses, microorganisms, or parasites is present in the edible oil composition of the liquid vaccine preparation in the form of a suspension.

Any appropriate liquid edible oil can be used as a liquid edible oil composition of a liquid vaccine preparation provided herein to maintain a desired percentage of a lyophilized vaccine component in the form of a suspension. For example, edible oils such as coconut oil, corn oil, cottonseed oil, olive oil, palm oil, peanut oil, rapeseed oil, safflower oil, sesame oil, soybean oil, or sunflower oil can be used as a liquid edible oil composition of a liquid vaccine preparation provided herein. In some cases, an edible oil containing one or more medium chain triglycerides can be used as a liquid edible oil composition of a liquid vaccine preparation provided herein. The fatty acids of such medium chain triglycerides can be caproic acid, caprylic acid, capric acid, or lauric acid. In some cases, at least 40 percent (e.g., at least 45 percent, at least 55 percent, at least 60 percent, at least 65 percent, at least 70 percent, at least 75 percent, at least 80 percent, at least 85 percent, at least 90 percent, at least 95 percent, or at least 99 percent) of the edible oil of a liquid vaccine preparation provided herein can be medium chain triglycerides. In some cases, the edible oil of a liquid vaccine preparation provided herein can be 100 percent medium chain triglycerides. In some cases, refined edible oils can be used as a liquid edible oil composition of a liquid vaccine preparation provided herein.

Any appropriate method can be used to obtain a liquid edible oil composition. For example, standard oil extraction and refinement techniques can be used to extract oil from plant sources such as coconuts, corn, cottonseed, olives, peanuts, rapeseeds, soybeans, or sunflowers. In some cases, oil extraction and refinement techniques such as those described in U.S. Pat. Nos. 4,243,603; 4,255,346; 4,623,489; 5,932,261; and 7,531,678 can be used to make liquid edible oil. In some cases, liquid edible oils can be obtained commercially from suppliers such as ADM (Decatur, Ill.), Bunge North America (St. Louis, Mo.), Monsanto Company (St. Louis, Mo.), or Stepan Company (Northfield, Ill.).

A liquid vaccine preparation provided herein can be designed for oral delivery to a mammal (e.g., human) and can include a liquid edible oil composition and a lyophilized, viable vaccine component present in the liquid edible oil composition in the form of a suspension. The ability to maintain at least 90 percent of a lyophilized vaccine component present in the liquid edible oil composition of a liquid vaccine preparation provided herein in the form of a suspension can allow the viability of the lyophilized vaccine component to remain high for extended periods of time (e.g., at least one, two, three, four, five, six months or more) without requiring the liquid vaccine preparation to be stored at temperatures below 8° C. (e.g., below 7° C., 6° C., 5° C., 4° C., 3° C., or 2° C.). For example, a liquid vaccine preparation provided herein can exhibit no more than a 50 percent (e.g., no more than a 45 percent, no more than a 40 percent, no more than a 35 percent, no more than a 30 percent, no more than a 25 percent, no more than a 20 percent, no more than a 15 percent, no more than a 10 percent, no more than a 5 percent, or no more than a 1 percent) reduction in viability when stored at 37° C. for 30 days. In some cases, a liquid vaccine preparation provided herein can exhibit no more than a 50 percent (e.g., no more than a 45 percent, no more than a 40 percent, no more than a 35 percent, no more than a 30 percent, no more than a 25 percent, no more than a 20 percent, no more than a 15 percent, no more than a 10 percent, no more than a 5 percent, or no more than a 1 percent) reduction in viability when stored at 40° C. or 45° C. for 30 days. In some cases, a liquid vaccine preparation provided herein can exhibit no more than a 50 percent (e.g., no more than a 45 percent, no more than a 40 percent, no more than a 35 percent, no more than a 30 percent, no more than a 25 percent, no more than a 20 percent, no more than a 15 percent, no more than a 10 percent, no more than a 5 percent, or no more than a 1 percent) reduction in viability when stored at 37° C., 40° C., or 45° C. for at least 45 days, at least 60 days, or at least six months. The viability of a vaccine component can be determined using standard titer or viability determinations appropriate for the particular component being tested. For example, standard viral titer determinations can be used to assess the titer before and after storage at 37° C. for 30 days for a liquid vaccine preparation containing viable viruses. When a liquid vaccine preparation contains viable bacteria, standard measurement techniques designed to assess bacteria viability can be used.

In some cases, a liquid vaccine preparation provided herein can be designed to include a liquid edible oil composition and a lyophilized vaccine component (e.g., viable viruses) present in the liquid edible oil composition in the form of a suspension. Such a liquid vaccine preparation can have any viral titer appropriate for vaccination purposes. For example, such a liquid vaccine preparation can have a viral titer of at least 1×10⁴ (e.g., at least 2.5×10⁴, at least 5×10⁴, at least 1×10⁵, at least 5×10⁵, at least 1×10⁶, at least 5×10⁶, or at least 1×10⁷) PFU or FFU after being stored at 37° C. for 30 days. In some cases, such a liquid vaccine preparation can have a viral titer of at least 1×10⁴ (e.g., at least 2.5×10⁴, at least 5×10⁴, at least 1×10⁵, at least 5×10⁵, at least 1×10⁶, at least 5×10⁶, or at least 1×10⁷) PFU or FFU after being stored at 40° C. or 45° C. for 30 days. In some cases, such a liquid vaccine preparation can have a viral titer of at least 1×10⁴ (e.g., at least 2.5×10⁴, at least 5×10⁴, at least 1×10⁵, at least 5×10⁵, at least 1×10⁶, at least 5×10⁶, or at least 1×10⁷) PFU or FFU after being stored at 37° C., 40° C., or 45° C. for at least 45 days, at least 60 days, or at least six months.

In some cases, a liquid vaccine preparation provided herein can be designed to include a liquid edible oil composition and a lyophilized vaccine component of viable microorganisms present in the liquid edible oil composition in the form of a suspension. Such a liquid vaccine preparation can have any concentration of viable microorganisms appropriate for vaccination purposes. For example, such a liquid vaccine preparation can have a viable microbial concentration of at least 1×10⁴ (e.g., at least 2.5×10⁴, at least 5×10⁴, at least 1×10⁵, at least 5×10⁵, at least 1×10⁶, at least 5×10⁶, or at least 1×10⁷) CFU after being stored at 37° C. for 30 days. In some cases, such a liquid vaccine preparation can have a viable microbial concentration of at least 1×10⁴ (e.g., at least 2.5×10⁴, at least 5×10⁴, at least 1×10⁵, at least 5×10⁵, at least 1×10⁶, at least 5×10⁶, or at least 1×10⁷) CFU after being stored at 40° C. or 45° C. for 30 days. In some cases, such a liquid vaccine preparation can have a viable microbial concentration of at least 1×10⁴ (e.g., at least 2.5×10⁴, at least 5×10⁴, at least 1×10⁵, at least 5×10⁵, at least 1×10⁶, at least 5×10⁶, or at least 1×10⁷) CFU after being stored at 37° C., 40° C., or 45° C. for at least 45 days, at least 60 days, or at least six months.

A liquid vaccine preparation provided herein can include any appropriate volume of a liquid edible oil composition and any appropriate amount of a vaccine component. For example, a liquid vaccine preparation provided herein can be composed of between about 0.1 mL and about 10 mL (e.g., between about 0.1 mL and about 5 mL, between about 0.1 mL and about 2.5 mL, between about 0.5 mL and about 10 mL, between about 1 mL and about 10 mL, between about 1 mL and about 5 mL, between about 1 mL and about 2.5 mL, or between about 1 mL and about 2 mL) of a liquid edible oil composition. In some cases, a liquid vaccine preparation provided herein can be composed of between about 10 μg and about 1 g (e.g., between about 50 μg and about 1 g, between about 100 μg and about 1 g, between about 100 μg and about 750 mg, between about 100 μg and about 500 mg, or between about 500 μg and about 250 mg) of a vaccine component (e.g., a lyophilized vaccine component). The amount of vaccine component present within a liquid vaccine preparation provided herein can be an amount sufficient to provide the liquid vaccine preparation with between about 5×10⁴ and about 5×10⁹ (e.g., between about 1×10⁵ and about 1×10⁸, between about 2.5×10⁵ and about 1×10⁸, between about 5×10⁵ and about 1×10⁸, between about 1×10⁶ and about 1×10⁸, or between about 5×10⁵ and about 1×10⁷) PFUs or FFUs for viruses or CFUs for microorganisms. In some cases, the amount of vaccine component present within a liquid vaccine preparation provided herein can be an amount sufficient to provide the liquid vaccine preparation with between about 1×10³ and about 1×10⁸ (e.g., between about 1×10⁵ and about 1×10⁸, between about 2.5×10⁵ and about 1×10⁸, between about 5×10⁵ and about 1×10⁸, between about 1×10⁶ and about 1×10⁸, or between about 5×10⁵ and about 1×10⁷) viable parasites/mL for an oral vaccine preparation designed to immunize a mammal against a parasite.

A liquid vaccine preparation provided herein can include a buffer component that is present, at least in part, in an edible oil composition of the liquid vaccine preparation as a suspension. In some cases, no more than 5 percent (e.g., no more than 4 percent, no more than 3 percent, no more than 2 percent, or no more than 1 percent) of a buffer component is dissolved in the edible oil composition of the liquid vaccine preparation. In some cases, at least 90 percent (e.g., at least 91 percent, at least 92 percent, at least 93 percent, at least 94 percent, at least 95 percent, at least 96 percent, at least 97 percent, at least 98 percent, or at least 99 percent) of a buffer component is present in the edible oil composition of the liquid vaccine preparation in the form of a suspension. Having the ability to maintain both a vaccine component and a buffer component in the form of suspensions within an edible oil composition of a liquid vaccine preparation provided herein can allow both to be present within the same liquid vaccine preparation without interacting with each other.

Any appropriate buffer can be used as a buffer component of a liquid vaccine preparation provided herein. For example, the buffer component can include calcium carbonate, sodium carbonate, potassium carbonate, sodium bicarbonate, potassium bicarbonate, calcium bicarbonate, aluminum hydroxide, magnesium hydroxide, or any mixture thereof. In some cases, a buffer component can be a powdered buffer component. A buffer component of a liquid vaccine preparation provided herein can be used to reduce the effects a gastric fluid on the vaccine component, thereby increasing the viability of the vaccine component when the liquid vaccine preparation is orally administered to a mammal.

A liquid vaccine preparation provided herein can include any appropriate amount of a buffer component. For example, a liquid vaccine preparation provided herein can be composed of between about 1 mg and about 5 gram (e.g., between about 1 mg and about 2.5 gram, between about 10 mg and about 750 mg, between about 20 mg and about 750 mg, between about 50 mg and about 750 mg, between about 100 mg and about 750 mg, between about 10 mg and about 500 mg, or between about 10 mg and about 250 mg) of a buffer component (e.g., a powdered form of a buffer component). In some cases, a liquid vaccine preparation provided herein can be composed of about 60 mg of a buffer component (e.g., a powdered form of a buffer component). The amount of buffer component present within a liquid vaccine preparation provided herein can be an amount that sufficiently reduces the effects of gastric fluid on the vaccine components such that an effective amount of viable vaccine component is delivered to a mammal upon oral administration.

A liquid vaccine preparation provided herein can include other ingredients such as fillers, colorants, flavorings, sweeteners, thickeners, stabilizers, or combinations thereof. In general, such other ingredients can be included in a liquid vaccine preparation provided that they do not substantially reduce the stability of a vaccine component and provided that they do not substantially increase the solubility of a vaccine component, a buffer component, or both within an edible oil of the liquid vaccine preparation. For example, other ingredients that can be included within a liquid vaccine preparation provided herein included, without limitation, those that lack water.

A liquid vaccine preparation provided herein configured into a delivery device containing a single dosage amount or multiple dosage amounts. For example, a delivery device such as a syringe, blister pack, plastic squeeze tube or bottle, or a glass vial with a separate delivery device can be configured to house a single unit dosage amount of a liquid vaccine preparation provided herein. Any appropriate amount of a liquid vaccine preparation provided herein can be a unit dosage amount for oral delivery to a mammal. In general, between about 0.1 mL and about 10 mL (e.g., between about 0.1 mL and about 5 mL, between about 0.1 mL and about 2.5 mL, between about 0.5 mL and about 10 mL, between about 1 mL and about 10 mL, between about 1 mL and about 5 mL, between about 1 mL and about 2.5 mL, or between about 1 mL and about 2 mL) can be a unit dosage amount of a liquid vaccine preparation provided herein for oral delivery to a mammal.

In some cases, a liquid vaccine preparation provided herein can include a polypeptide preparation as a vaccine component. For example, such a vaccine component can be a ricin polypeptide, an Aβ peptide, or other polypeptide. In such cases, the liquid vaccine preparation containing a polypeptide preparation as the vaccine component can exhibit no more than a 50 percent (e.g., no more than a 45 percent, no more than a 40 percent, no more than a 35 percent, no more than a 30 percent, no more than a 25 percent, no more than a 20 percent, no more than a 15 percent, no more than a 10 percent, no more than a 5 percent, or no more than a 1 percent) reduction in the ability to induce an immune response in a mammal when stored at 37° C. for 30 days. Any appropriate assays can be used to assess a composition's ability to induce an immune response in a mammal.

In the preceding description, particular embodiments may be described in isolation for clarity. Unless otherwise expressly specified that the features of a particular embodiment are incompatible with the features of another embodiment, certain embodiment can include a combination of compatible features described herein in connection with one or more embodiments.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., “between 1 to 5” includes 1, 1.5, 2, 2.75, 3, 3.80, 4, 5, etc.).

The invention will be further described in the following examples, which do not limit the scope of the invention described in the claims.

### EXAMPLES

### Example 1

### Stability of Quadrivalent Rotavirus Vaccine in Various Formulations Containing Edible Oil

Unit dosages (about 4×10⁵ PFUs) of a Rotavirus vaccine (RotaShield) produced by IDT Biologika were either lyophilized to form a dry composition or lyophilized and milled to form a powder. Pure medium chain triglyceride oil (NEOBEE M-5) was obtained from Stepan Company (Northfield, Ill.). For preparation A, one oral dose (about 4×10⁵ PFUs) of the lyophilized Rotavirus vaccine was combined with one mL of the pure medium chain triglyceride oil. For preparation B, one oral dose (about 4×10⁵ PFUs) of the lyophilized Rotavirus vaccine was combined with one mL of the pure medium chain triglyceride oil, 60 mg of powdered CaCO₃, and 5 μL of Tween-80. For preparation C, one oral dose (about 4×10⁵ PFUs) of the milled Rotavirus vaccine powder was combined with one mL of the pure medium chain triglyceride oil. For preparation D, one oral dose (about 4×10⁵ PFUs) of the milled Rotavirus vaccine powder was combined with one mL of the pure medium chain triglyceride oil, 60 mg of powdered CaCO₃, and 5 μl, of Tween-80.

The viral titers for preparations A, B, C, and D were initially determined and were determined after being stored for 30 days at either 4° C. or 37° C., stored for 60 days at either 4° C., 25° C. or 37° C., or stored for six months at either 4° C., 25° C. or 37° C. To determine viral titers, a preparation was mixed with 1 mL of pure water with (for preparation A and C) or without (for preparations B and D) 0.1 percent Tween-20. The vials containing the mixtures were vortexed, and the mixtures were transferred to 50 mL conical tubes and sonicated for two rounds of sonication at an output of 5 for 10 seconds. Each vaccine formulation formed an emulsion following sonication. The conical tubes were centrifuged at 4000 rpm for 5 minutes. After centrifugation, three layers were visible. The bottom layer was a concentrated solid (most likely CaCO₃). The middle layer was a pink aqueous liquid (most likely a portion of the vaccine viruses). The upper layer was a pink/white foamy liquid (most likely the oil along with some residual other ingredients). The aqueous layer was isolated and used to determine FFU/mL for each vaccine formulation at the initial time point and after 30 days of storage at 4° C. or 37° C. (Table 1), after 60 days of storage at 4° C., 25° C., or 37° C. (Table 2), and after six months of storage at 4° C., 25° C., or 37° C. (Table 2).

TABLE 1
Titer determinations for preparations stored at 4° C. or 37° C. for 30 days.

| | | Initial Titer | | |
|---|---|---|---|---|
| | | FFU/mL | Storage | Titer after 30 |
| Preparation | Contents | (log₁₀)^{a} | Temperature | days |
| A | Lyophilized vaccine + | 1.41 × 10⁶ (6.15) | 4° C. | 8.40 × 10⁵ (5.92) |
| | MCT oil | | 37° C. | 6.20 × 10⁵ (5.79) |
| B | Lyophilized vaccine + | 8.40 × 10⁵ (5.92) | 4° C. | 8.67 × 10⁵ (5.94) |
| | MCT oil + CaCO3 + | | 37° C. | 8.47 × 10⁵ (5.93) |
| | Tween-80 | | | |
| C | Milled lyophilized | 6.60 × 10⁵ (5.82) | 4° C. | nd |
| | vaccine powder + MCT | | 37° C. | nd |
| | oil | | | |
| D | Milled lyophilized | 3.33 × 10⁵ (5.52) | 4° C. | 3.13 × 10⁵ (5.50) |
| | vaccine powder + MCT | | 37° C. | 1.07 × 10⁵ (5.03) |
| | oil + CaCO3 + Tween-80 | | | |

^{a}Titers were determined using a polyclonal antibody, thus giving an overall titer for the vaccine.
nd: not determined.

These results indicate that the vaccine is stable after storage for 30 days in the presence of buffer at elevated temperatures (e.g. 37° C.) when stored as a suspension in an edible oil.

TABLE 2
Titer determinations for preparations stored at 4° C., 25° C., or 37° C.

| | | Initial Titer | | | |
|---|---|---|---|---|---|
| | | FFU/mL | Storage | | Titer after six |
| Preparation | Contents | (log₁₀)^{a} | Temp. | Titer after 60 days | months |
| A | Lyophilized | 1.41 × 10⁶ (6.15) | 4° C. | 1.37 × 10⁶ (6.14) | 1.57 × 10⁶ (6.19) |
| | vaccine + | | 25° C. | 1.04 × 10⁶ (6.02) | 2.06 × 10⁶ (6.31) |
| | MCT oil | | 37° C. | 3.53 × 10⁵ (5.55) | 6.87 × 10⁵ (5.84) |
| B | Lyophilized | 8.40 × 10⁵ (5.92) | 4° C. | 1.59 × 10⁶ (6.20) | 1.09 × 10⁶ (6.04) |
| | vaccine + | | 25° C. | 1.23 × 10⁶ (6.09) | 1.47 × 10⁵ (5.17) |
| | MCT oil + | | 37° C. | 6.13 × 10⁵ (5.79) | 2.37 × 10⁵ (5.38) |
| | CaCO3 + | | | | |
| | Tween-80 | | | | |
| C | Milled | 6.60 × 10⁵ (5.82) | 4° C. | 4.33 × 10⁵ (5.64) | 1.01 × 10⁶ (6.00) |
| | lyophilized | | 25° C. | 3.41 × 10⁵ (5.53) | 6.00 × 10⁵ (5.78) |
| | vaccine | | 37° C. | nd | nd |
| | powder + | | | | |
| | MCT oil | | | | |
| D | Milled | 3.33 × 10⁵ (5.52) | 4° C. | 4.53 × 10⁵ (5.66) | 2.91 × 10⁵ (5.46) |
| | lyophilized | | 25° C. | 2.61 × 10⁵ (5.42) | 1.09 × 10⁵ (5.04) |
| | vaccine | | 37° C. | nd | nd |
| | powder + | | | | |
| | MCT oil + | | | | |
| | CaCO3 + | | | | |
| | Tween-80 | | | | |

^{a}Titers were determined using a polyclonal antibody, thus giving an overall titer for the vaccine.
nd: not determined.

These results indicate that the vaccine is stable after storage for 60 days in the presence of buffer at elevated temperatures (e.g. 25° C. or 37° C.) when stored as a suspension in an edible oil.

### Example 23

### Making a Shelf-Stable, Liquid, Oral Rotavirus Vaccine

MTC oil is added to a vessel as is an appropriated amount of HUBERCAL Elite 950 CaCO₃ and an appropriate amount of lyophilized RotaShield powder to provide an appropriate ratio of MCT (1.0 mL)/CaCO₃ (60 mg)/RotaShield (4×10⁵ PFU) in the equivalent of about 1.0 mL final volume. The material is stirred until a uniform suspension is achieved. The suspension is piped into packaging equipment for an aseptic fill operation.

### Example 3

### Making a Shelf-Stable, Liquid, Oral Rotavirus Vaccine

An appropriated amount of HUBERCAL Elite 950 CaCO₃ is added to a blending vessel as is an appropriate amount of lyophilized RotaShield powder to provide an appropriate ratio of CaCO₃/RotaShield in a final dosage. Material is dry blended until a uniform blend is achieved. The powder blend is transferred to the packaging line. One vaccine dose (dry) is filled into a final container, and MCT oil (about 1.0 mL) is added to the same container, which is then sealed.

### Example 4

### Making a Shelf-Stable, Liquid, Oral Vaccine for Cholera

A Cholera Vaccine (e.g., DUKORAL) is lyophilized using standard freeze drying equipment, and one dose of the dry vaccine is added to 5 mL of MCT oil. Dry excipients such as sodium dihydrogen phosphate monohydrate (about 1.7 mg), disodium hydrogen phosphate dehydrate (about 9.4 mg), sodium chloride (about 26 mg), sodium hydrogen carbonate (about 3600 mg), sodium carbonate anhydrous (about 400 mg), saccharin sodium (about 30 mg), and sodium citrate (about 6 mg) is optionally added, and the final product is packaged.

### Example 5

### Making a Shelf-Stable, Liquid, Oral Vaccine for Polioviruses

A polio vaccine is lyophilized using standard freeze drying equipment or as described elsewhere (Shiomi et al., Japan J. Infect. Dis., 56:70-72 (2003)). One dose of the dry vaccine is added to 5 mL of MCT oil, and the final product is packaged.

### EMBODIMENTS

### Embodiment 1

A liquid vaccine preparation for oral delivery to a mammal, wherein said preparation comprises an edible oil in liquid form and viable viruses, microorganisms, or parasites present within said edible oil in the form of a suspension, wherein said vaccine preparation undergoes no more than a 50 percent reduction in viability of said viable viruses, microorganisms, or parasites when stored at 37° C. for 30 days.

### Embodiment 2

The vaccine preparation of embodiment 1, wherein said preparation comprises viable viruses.

### Embodiment 3

The vaccine preparation of any preceding embodiment, wherein said preparation comprises viable microorganisms.

### Embodiment 4

The vaccine preparation of any preceding embodiment, wherein said preparation comprises viable parasites.

### Embodiment 5

The vaccine preparation of any preceding embodiment, wherein said preparation comprises a dry or lyophilized viable viruses, microorganisms, or parasites.

### Embodiment 6

The vaccine preparation of any preceding embodiment, wherein said preparation comprises a buffer component present within said edible oil in the form of a suspension.

### Embodiment 7

The vaccine preparation of any preceding embodiment, wherein said vaccine preparation undergoes no more than a 25 percent reduction in viability of said viable viruses, microorganisms, or parasites when stored at 37° C. for 30 days.

### Embodiment 8

The vaccine preparation of any preceding embodiment, wherein said vaccine preparation undergoes no more than a 10 percent reduction in viability of said viable viruses, microorganisms, or parasites when stored at 37° C. for 30 days.

### Embodiment 9

The vaccine preparation of any preceding embodiment, wherein said vaccine preparation undergoes no more than a 5 percent reduction in viability of said viable viruses, microorganisms, or parasites when stored at 37° C. for 30 days.

### Embodiment 10

The vaccine preparation of any preceding embodiment, wherein said vaccine preparation undergoes no more than a 1 percent reduction in viability of said viable viruses, microorganisms, or parasites when stored at 37° C. for 30 days.

### Embodiment 11

A vaccine preparation comprising an edible oil in liquid form, a buffer component present within said edible oil in the form of a suspension, and viable viruses present within said edible oil in the form of a suspension, wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

### Embodiment 12

The vaccine preparation of embodiment 11, wherein said edible oil comprises medium chain triglycerides.

### Embodiment 13

The vaccine preparation of embodiment 12, wherein fatty acids of said medium chain triglycerides comprise caproic acid, caprylic acid, capric acid, or lauric acid.

### Embodiment 14

The vaccine preparation of any one of embodiments 11-13, wherein more than 40 percent of said edible oil comprises medium chain triglycerides.

### Embodiment 15

The vaccine preparation of any one of embodiments 11-14, wherein more than 50 percent of said edible oil comprises medium chain triglycerides.

### Embodiment 16

The vaccine preparation of any one of embodiments 11-15, wherein more than 60 percent of said edible oil comprises medium chain triglycerides.

### Embodiment 17

The vaccine preparation of any one of embodiments 11-16, wherein more than 65 percent of said edible oil comprises medium chain triglycerides.

### Embodiment 18

The vaccine preparation of any one of embodiments 11-17, wherein said edible oil comprises a coconut oil, a corn oil, a cottonseed oil, an olive oil, a palm oil, a peanut oil, a rapeseed oil, a safflower oil, a sesame oil, a soybean oil, or a sunflower oil.

### Embodiment 19

The vaccine preparation of any one of embodiments 11-18, wherein said edible oil is an olive oil, a soybean oil, or a sunflower oil.

### Embodiment 20

The vaccine preparation of any one of embodiments 11-19, wherein said buffer component comprises calcium carbonate, sodium carbonate, potassium carbonate, sodium bicarbonate, potassium bicarbonate, calcium bicarbonate, aluminum hydroxide, or magnesium hydroxide.

### Embodiment 21

The vaccine preparation of any one of embodiments 11-20, wherein said buffer component is sodium bicarbonate or calcium carbonate.

### Embodiment 22

The vaccine preparation of any one of embodiments 11-21, wherein said viable viruses are attenuated rotaviruses.

### Embodiment 23

The vaccine preparation of any one of embodiments 11-22, wherein said viable viruses are a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4.

### Embodiment 24

The vaccine preparation of any one of embodiments 11-23, wherein said viable viruses are a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4.

### Embodiment 25

The vaccine preparation of any one of embodiments 11-24, wherein said viable viruses are attenuated polioviruses.

### Embodiment 26

The vaccine preparation of any one of embodiments 11-25, wherein said viable viruses comprise lyophilized viable viruses.

### Embodiment 27

The vaccine preparation of any one of embodiments 11-26, wherein said vaccine preparation has a viral titer of at least 3.5×10⁵ PFUs or FFUs per mL of vaccine preparation.

### Embodiment 28

The vaccine preparation of any one of embodiments 11-27, wherein said vaccine preparation has a viral titer of at least 8×10⁵ PFUs or FFUs per mL of vaccine preparation.

### Embodiment 29

The vaccine preparation of any one of embodiments 11-28, wherein said vaccine preparation lacks water.

### Embodiment 30

The vaccine preparation of any one of embodiments 11-29, wherein said viable viruses of said vaccine preparation were lyophilized viable viruses that were combined with said edible oil.

### Embodiment 31

The vaccine preparation of any one of embodiments 11-30, wherein said buffer component of said vaccine preparation was a powder form of said buffer component that was combined with said edible oil.

### Embodiment 32

The vaccine preparation of any one of embodiments 11-31, wherein said vaccine preparation lacks dissolved viable viruses.

### Embodiment 33

The vaccine preparation of any one of embodiments 11-32, wherein said vaccine preparation lacks a dissolved buffer component.

### Embodiment 34

The vaccine preparation of any one of embodiments 11-33, wherein said vaccine preparation lacks dissolved viable viruses and a dissolved buffer component.

### Embodiment 35

The vaccine preparation of any one of embodiments 11-34, wherein said vaccine preparation comprises a colorant.

### Embodiment 36

The vaccine preparation of any one of embodiments 11-35, wherein said vaccine preparation comprises a colorant, flavoring, or sweetener.

### Embodiment 37

The vaccine preparation of any one of embodiments 11-36, wherein said vaccine preparation is in the form of a liquid containing a suspension and adapted for oral administration.

### Embodiment 38

A method for making a vaccine preparation in liquid form, wherein said method comprises combining dry viable viruses with an edible oil in liquid form under conditions wherein said dry viable viruses form a suspension within said edible oil, thereby forming said vaccine preparation in liquid form, and wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

### Embodiment 39

The method of embodiment 38, wherein said dry viable viruses are dry attenuated rotaviruses.

### Embodiment 40

The method of embodiment 38 or embodiment 39, wherein said dry viable viruses are a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4.

### Embodiment 41

The method of any one of embodiments 38-40, wherein said viable viruses are a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4.

### Embodiment 42

The method of any one of embodiments 38-41, wherein said dry viable viruses are dry attenuated polioviruses.

### Embodiment 43

The method of any one of embodiments 38-42, wherein said dry viable viruses comprise lyophilized viable viruses.

### Embodiment 44

The method of any one of embodiments 38-43, wherein said vaccine preparation has a viral titer of at least 3.5×10⁵ PFUs or FFUs per mL of vaccine preparation.

### Embodiment 45

The method of any one of embodiments 38-44, wherein said vaccine preparation has a viral titer of at least 8×10⁵ PFUs or FFUs per mL of vaccine preparation.

### Embodiment 46

The method of any one of embodiments 38-45, wherein said edible oil comprises medium chain triglycerides.

### Embodiment 47

The method of embodiment 46, wherein fatty acids of said medium chain triglycerides comprise caproic acid, caprylic acid, capric acid, or lauric acid.

### Embodiment 48

The method of any one of embodiments 38-47, wherein more than 40 percent of said edible oil comprises medium chain triglycerides.

### Embodiment 49

The method of any one of embodiments 38-48, wherein more than 50 percent of said edible oil comprises medium chain triglycerides.

### Embodiment 50

The method of any one of embodiments 38-49, wherein more than 60 percent of said edible oil comprises medium chain triglycerides.

### Embodiment 51

The method of any one of embodiments 38-50, wherein more than 65 percent of said edible oil comprises medium chain triglycerides.

### Embodiment 52

The method of any one of embodiments 38-51, wherein said edible oil comprises a coconut oil, a corn oil, a cottonseed oil, an olive oil, a palm oil, a peanut oil, a rapeseed oil, a safflower oil, a sesame oil, a soybean oil, or a sunflower oil.

### Embodiment 53

The method of any one of embodiments 38-52, wherein said edible oil is an olive oil, a soybean oil, or a sunflower oil.

### Embodiment 54

The method of any one of embodiments 38-53, wherein said method comprises combining a dry buffer component with said edible oil under conditions wherein said dry buffer component forms a suspension within said edible oil.

### Embodiment 55

The method of embodiment 54, wherein said dry buffer component comprises calcium carbonate, sodium carbonate, potassium carbonate, sodium bicarbonate, potassium bicarbonate, calcium bicarbonate, aluminum hydroxide, magnesium hydroxide, or a mixture thereof.

### Embodiment 56

The method of embodiment 54 or embodiment 55, wherein said buffer component is sodium bicarbonate, calcium carbonate, or a mixture thereof.

### Embodiment 57

The method of any one of embodiments 54-56, wherein said buffer component is in powder form prior to combining said dry buffer component with said edible oil.

### Embodiment 58

The method of any one of embodiments 38-57, wherein said vaccine preparation lacks water.

### Embodiment 59

The method of any one of embodiments 38-58, wherein said method lacks a step of adding water to said vaccine preparation.

### Embodiment 60

The method of any one of embodiments 38-59, wherein said vaccine preparation lacks dissolved viable viruses.

### Embodiment 61

The method of any one of embodiments 38-60, wherein said vaccine preparation lacks a dissolved buffer component.

### Embodiment 62

The method of any one of embodiments 38-61, wherein said vaccine preparation lacks dissolved viable viruses and a dissolved buffer component.

### Embodiment 63

The method of any one of embodiments 38-62, wherein said method comprises combining a colorant with said edible oil.

### Embodiment 64

The method of any one of embodiments 38-63, wherein said method comprises combining flavoring or sweetener with said edible oil.

### Embodiment 65

The method of any one of embodiments 38-64, wherein said method comprises inserting said vaccine preparation in the form of a liquid containing a suspension into a container adapted for oral delivery to a mammal.

### Embodiment 66

The method of any one of embodiments 38-65, wherein said container is an oral syringe.

### Embodiment 67

The method of any one of embodiments 38-66, wherein said container is an oral syringe-like container.

### Embodiment 68

The method of any one of embodiments 38-67, wherein said mammal is a human.

### Embodiment 69

The method of any one of embodiments 38-68, wherein said mammal is a human child no more than three years of age.

### Embodiment 70

A method for vaccinating a mammal, wherein said method comprises orally administering a vaccine preparation in liquid form to a mammal, wherein said vaccine preparation comprises an edible oil in liquid form and viable viruses present within said edible oil in the form of a suspension, wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

### Embodiment 71

The method of embodiment 70, wherein said edible oil comprises medium chain triglycerides.

### Embodiment 72

The method of embodiment 71, wherein fatty acids of said medium chain triglycerides comprise caproic acid, caprylic acid, capric acid, or lauric acid.

### Embodiment 73

The method of any one of embodiments 70-72, wherein more than 40 percent of said edible oil comprises medium chain triglycerides.

### Embodiment 74

The method of any one of embodiments 70-73, wherein more than 50 percent of said edible oil comprises medium chain triglycerides.

### Embodiment 75

The method of any one of embodiments 70-74, wherein more than 60 percent of said edible oil comprises medium chain triglycerides.

### Embodiment 76

The method of any one of embodiments 70-75, wherein more than 65 percent of said edible oil comprises medium chain triglycerides.

### Embodiment 77

The method of any one of embodiments 70-76, wherein said edible oil comprises a coconut oil, a corn oil, a cottonseed oil, an olive oil, a palm oil, a peanut oil, a rapeseed oil, a safflower oil, a sesame oil, a soybean oil, a sunflower oil, or a mixture thereof.

### Embodiment 78

The method of any one of embodiments 70-77, wherein said edible oil is an olive oil, a soybean oil, a sunflower oil, or a mixture thereof.

### Embodiment 79

The method of any one of embodiments 70-78, wherein said vaccine preparation comprises a buffer component present within said edible oil in the form of a suspension.

### Embodiment 80

The method of embodiment 79, wherein said buffer component comprises calcium carbonate, sodium carbonate, potassium carbonate, sodium bicarbonate, potassium bicarbonate, calcium bicarbonate, aluminum hydroxide, magnesium hydroxide, or a mixture thereof.

### Embodiment 81

The method of embodiment 79 or embodiment 80, wherein said buffer component is sodium bicarbonate, calcium carbonate, or a mixture thereof.

### Embodiment 82

The method of any one of embodiments 79-81, wherein said buffer component of said vaccine preparation was a powder form of said buffer component that was combined with said edible oil.

### Embodiment 83

The method of any one of embodiments 70-82, wherein said viable viruses are attenuated rotaviruses.

### Embodiment 84

The method of any one of embodiments 70-83, wherein said viable viruses are a rhesus rotavirus serotype 3, a rhesus-human VP7 reassortant rotavirus serotype 1, a rhesus-human VP7 reassortant rotavirus serotype 2, or a rhesus-human VP7 reassortant rotavirus serotype 4.

### Embodiment 85

The method of any one of embodiments 70-84, wherein said viable viruses are a rhesus rotavirus serotype 3, a rhesus-human VP7 reassortant rotavirus serotype 1, a rhesus-human VP7 reassortant rotavirus serotype 2, and a rhesus-human VP7 reassortant rotavirus serotype 4.

### Embodiment 86

The method of any one of embodiments 70-85, wherein said viable viruses are attenuated polioviruses.

### Embodiment 87

The method of any one of embodiments 70-86, wherein said viable viruses comprise lyophilized viable viruses.

### Embodiment 88

The method of any one of embodiments 70-87, wherein said vaccine preparation has a viral titer of at least 3.5×10⁵ PFUs or FFUs per mL of vaccine preparation.

### Embodiment 89

The method of any one of embodiments 70-88, wherein said vaccine preparation has a viral titer of at least 8×10⁵ PFUs or FFUs per mL of vaccine preparation.

### Embodiment 90

The method of any one of embodiments 70-89, wherein said vaccine preparation lacks water.

### Embodiment 91

The method of any one of embodiments 70-90, wherein said viable viruses of said vaccine preparation were lyophilized viable viruses that were combined with said edible oil.

### Embodiment 92

The method of any one of embodiments 70-91, wherein said vaccine preparation lacks dissolved viable viruses.

### Embodiment 93

The method of any one of embodiments 70-92, wherein said vaccine preparation lacks a dissolved buffer component.

### Embodiment 94

The method of any one of embodiments 70-93, wherein said vaccine preparation lacks dissolved viable viruses and a dissolved buffer component.

### Embodiment 95

The method of any one of embodiments 70-94, wherein said vaccine preparation comprises a colorant.

### Embodiment 96

The method of any one of embodiments 70-95, wherein said vaccine preparation comprises flavoring or sweetener.

### Embodiment 97

The method of any one of embodiments 70-96, wherein said mammal is a human.

### Embodiment 98

The method of any one of embodiments 70-97, wherein said mammal is a human child no more than three years of age.

### Embodiment 99

The method of any one of embodiments 70-98, wherein said vaccine preparation was stored at room temperature for a period of time before being administered to said mammal, wherein said period of time is at least 15 days.

### Embodiment 100

The method of embodiment 99, wherein said period of time is at least 30 days.

### Embodiment 101

The method of any one of embodiments 70-100, wherein said vaccine preparation was stored at a temperature between 15° C. and 30° C. for a period of time before being administered to said mammal, wherein said period of time is at least five days.

### Embodiment 102

The method of embodiment 101, wherein said period of time is at least ten days.

### Embodiment 103

The method of embodiment 101 or embodiment 102, wherein said period of time is at least 30 days.

### Embodiment 104

The method of any one of embodiments 101-103, wherein said temperature is between 18° C. and 25° C.

### Embodiment 105

The method of embodiment 104, wherein said period of time is at least ten days.

### Embodiment 106

The method of embodiment 104 or embodiments 105, wherein said period of time is at least 30 days.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A liquid vaccine preparation for oral delivery to a mammal, wherein said preparation comprises an edible oil in liquid form and viable viruses, microorganisms, or parasites present within said edible oil in the form of a suspension, wherein said vaccine preparation undergoes no more than a 50 percent reduction in viability of said viable viruses, microorganisms, or parasites when stored at 37° C. for 30 days.

2. The vaccine preparation of claim 1, wherein said preparation comprises viable viruses.

3. The vaccine preparation of claim 1, wherein said preparation comprises viable microorganisms.

4. The vaccine preparation of claim 1, wherein said preparation comprises viable parasites.

5. The vaccine preparation of claim 1, wherein said preparation comprises a dry or lyophilized viable viruses, microorganisms, or parasites.

6. The vaccine preparation of claim 1, wherein said preparation comprises a buffer component present within said edible oil in the form of a suspension.

7-10. (canceled)

11. A vaccine preparation comprising an edible oil in liquid form, a buffer component present within said edible oil in the form of a suspension, and viable viruses present within said edible oil in the form of a suspension, wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

12. The vaccine preparation of claim 11, wherein said edible oil comprises medium chain triglycerides.

13. The vaccine preparation of claim 12, wherein fatty acids of said medium chain triglycerides comprise caproic acid, caprylic acid, capric acid, or lauric acid.

14-17. (canceled)

18. The vaccine preparation of claim 11, wherein said edible oil comprises a coconut oil, a corn oil, a cottonseed oil, an olive oil, a palm oil, a peanut oil, a rapeseed oil, a safflower oil, a sesame oil, a soybean oil, or a sunflower oil.

19. (canceled)

20. The vaccine preparation of claim 11, wherein said buffer component comprises calcium carbonate, sodium carbonate, potassium carbonate, sodium bicarbonate, potassium bicarbonate, calcium bicarbonate, aluminum hydroxide, or magnesium hydroxide.

21-37. (canceled)

38. A method for making a vaccine preparation in liquid form, wherein said method comprises combining dry viable viruses with an edible oil in liquid form under conditions wherein said dry viable viruses form a suspension within said edible oil, thereby forming said vaccine preparation in liquid form, and wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

39. The method of claim 38, wherein said dry viable viruses are dry attenuated rotaviruses.

40-41. (canceled)

42. The method of claim 38, wherein said dry viable viruses are dry attenuated polioviruses.

43. The method of claim 38, wherein said dry viable viruses comprise lyophilized viable viruses.

44-69. (canceled)

70. A method for vaccinating a mammal, wherein said method comprises orally administering a vaccine preparation in liquid form to a mammal, wherein said vaccine preparation comprises an edible oil in liquid form and viable viruses present within said edible oil in the form of a suspension, wherein (a) said vaccine preparation has a viral titer of at least 1×10⁵ PFUs or FFUs per mL of vaccine preparation or (b) said vaccine preparation undergoes no more than a 50 percent reduction in viability of said viable viruses when stored at 37° C. for 30 days.

71-96. (canceled)

97. The method of claim 70, wherein said mammal is a human.

98. The method of claim 70, wherein said mammal is a human child no more than three years of age.

99. The method of claim 70, wherein said vaccine preparation was stored at room temperature for a period of time before being administered to said mammal, wherein said period of time is at least 15 days.

100. (canceled)

101. The method of claim 70, wherein said vaccine preparation was stored at a temperature between 15° C. and 30° C. for a period of time before being administered to said mammal, wherein said period of time is at least five days.

102-106. (canceled)
